(19) United States
(12) Patent Application Publication
(10) Pub. No.: US 2025/0155347 A1
(43) Pub. Date: May 15, 2025

(54) POROUS SAMPLE WETTABILITY PARAMETER DETERMINING METHOD AND RELATED SYSTEM

(71) Applicant: TotalEnergies OneTech, COURBEVOIE (FR)

(72) Inventors: Mohamed REGAIEG, PAU CEDEX (FR); Régis BRUGIDOU, GURMENCON (FR); Franck NONO NGUENDJIO, PAU CEDEX (FR)

(73) Assignee: TotalEnergies OneTech, COURBEVOIE (FR)

(21) Appl. No.: 18/865,841

(22) PCT Filed: May 17, 2023
(86) PCT No.: PCT/EP2023/063270

§ 371 Date: Nov. 14, 2024

Related U.S. Application Data
(60) Provisional application No. 63/342,923, filed on May 17, 2022.

(51) Int. Cl. G01N 13/00; G01N 23/046; G01N 23/083; G06F 30/28

(52) U.S. Cl. CPC G01N 13/00; G01N 23/046; G01N 23/083; G06F 30/28; G01N 2223/401

(57) ABSTRACT

The method comprises saturating a porous sample with a first fluid, determining a first pore occupancy image of a first section of the porous sample.

It comprises carrying out a spontaneous imbibition from a first open face of the sample with a second fluid and determining a second pore occupancy image of the first section.

It further comprises saturating the porous sample with the second fluid and determining a third pore occupancy image of a second section of the porous sample.

The method comprises carrying out a spontaneous drainage with the first fluid from a second face of the sample and determining a fourth pore occupancy image of the second section.

The method comprises processing data extracted from the first, second, third and fourth pore occupancy images to determine a wettability parameter associated with the porous sample.

### CROSS REFERENCE TO RELATED APPLICATIONS

The present application is a U.S. National Phase Application under 35 U.S.C. § 371 of International Patent Application No. PCT/EP2023/063270 filed May 17, 2023, which claims priority of U.S. Provisional Patent Application No. 63/342,923 filed May 17, 2022. The entire contents of which are hereby incorporated by reference.

### FIELD OF THE INVENTION

The present invention concerns a porous sample wettability parameter determining method.

The invention applies in particular to the determination of crucial wettability parameters which are used in pore scale simulations.

### BACKGROUND

Several pore scale simulation techniques are used to simulate the flow in porous media such as Direct Numerical Simulation, Lattice Boltzmann method and Pore Network Modelling. Wettability is one of the main inputs of pore scale simulations. Unfortunately, it is difficult to characterize wettability parameters a priori. Moreover, even if a qualitative assignment of wettability is done and a first fluid-wet, second fluid-wet, or mixed-wet scenario is identified, a high number of uncertain parameters remains especially for the mixed-wet case as the contact angles, the fractions of first fluid-wet and second fluid-wet pores, wettability spatial correlations and wettability radii correlations are all important parameters that could affect the simulation results.

Determining wettability scheme of a porous medium/fluids system generally requires very long tests which can last in the order of magnitude of one year. Measurements of contact angles using multiphase micro-CT images have been tried. Nevertheless, the results are biased by image resolution, the technique is applicable only in the places where there is an interface, some of the measured menisci are pinned (not representative of the angles needed for the simulation) and sensitive to the way image processing is performed.

The assessment of the spatial correlation of wettability has also been obtained via measurements of contact angles using multiphase micro-CT images. Just as mentioned above, the results can be biased by the image resolution, and measurements are only possible in the places where there is an interface.

Assessment of the radii correlation of wettability has been done using a large number of SEM and ESEM images analysis. However, the technique is only qualitative and very time consuming.

Measurement of the water saturation at zero capillary pressure is also regularly carried out. These measurements however require a centrifuge Pc measurement or with other classical experiment. An extra experiment is needed, which complicates the method.

### SUMMARY

One aim of the invention is to obtain a very simple and time efficient process to determine a variety of wettability parameters to be used in pore scale simulation.

To this aim, the subject matter of the invention is a porous sample wettability parameter determining method, comprising the following steps:
placing a porous sample in a flow cell, the porous sample having a first open face and a second open face and an internal pore space;
saturating the porous sample with at least one first fluid until the at least one first fluid occupies a majority of the pore space;
taking a fluid sensitive first high resolution scan of at least one first section of the porous sample to determine a first pore occupancy image of the at least one first section of the porous sample;
after taking the first scan, carrying out a spontaneous imbibition from the first open face with a least one second fluid for a first given time;
taking a fluid sensitive second high resolution scan of the at least one first section of the porous sample to determine a second pore occupancy image of the at least one first section of the porous sample;
after taking the second scan, saturating the porous sample with the at least one second fluid until the at least one second fluid occupies at least a majority of the pore space;
taking a fluid sensitive third high resolution scan of at least one second section of the porous sample to determine a third pore occupancy image of the at least one second section of the porous sample;
after taking the third scan, carrying out a spontaneous drainage with the first fluid from the second open face for a second given time,
taking a fluid sensitive fourth high resolution scan of the at least one second section of the porous sample to determine a fourth pore occupancy image of the at least one second section of the porous sample;
processing data extracted from the first pore occupancy image, the second pore occupancy image, the third pore occupancy image and the fourth pore occupancy image to determine at least a wettability parameter associated with the porous sample.

The method according to the invention may comprises one or more of the following feature(s), taken solely or according to any technical feasible combination:
carrying out the spontaneous imbibition of the first open face with a second fluid comprises leaching the second fluid along the first open face without forcing the second fluid in the porous sample, carrying out a spontaneous drainage of the first open face with the first fluid comprising leaching the first fluid along the second open face without forcing the first fluid in the porous sample;
the first open face and the second open face are opposite faces of the porous sample or are a same face of the porous sample;
taking a fluid sensitive first, second, third and fourth high resolution scan comprises taking a computed tomography image or an x-Ray image of a section of the porous sample in particular in the micron or submicron resolution range;
taking a fluid sensitive first high resolution scan of at least one first section of the porous sample comprises taking several fluid sensitive first high resolution scans of several successive first sections of the porous sample along at least a first end region of the porous sample, taking a fluid sensitive second high resolution scan of the at least one first section of the porous sample comprises taking several fluid sensitive second high resolution scans of several successive first sections of the porous sample along at least the first end region of the porous sample;
taking a fluid sensitive third high resolution scan of at least one second section of the porous sample comprises taking several fluid sensitive third high resolution scans of several successive second sections of the porous sample along at least a second end region of the porous sample, taking a fluid sensitive fourth high resolution scan of the at least one second section of the porous sample comprises taking several fluid sensitive fourth high resolution scans of several successive second sections of the porous sample along at least the second end region of the porous sample;
the at least one first section is located in the vicinity of the first open face, the at least one second section being located in the vicinity of the second open face;
the difference between the first given time and the second given time is smaller than 10%;
the data processing includes calculating a saturation in first fluid or/and in second fluid in the first pore occupancy image, the second pore occupancy image, the third pore occupancy image and the fourth pore occupancy image;
the data processing comprises calculating a difference between a saturation in first fluid or/and in second fluid in the second pore occupancy image and the corresponding saturation in first fluid or/and in second fluid in the first pore occupancy image, and/or the data processing comprises calculating a difference between a saturation in first fluid or/and in second fluid in the fourth pore occupancy image and the corresponding saturation in first fluid or/and in second fluid in the third pore occupancy image;
the data processing includes calculating a pore size distribution as a function of pore size and/or a pore distribution of pores occupied by the first fluid as a function of pore size, and/or pore distribution of pores occupied by the second fluid as a function of pore size, in the first pore occupancy image, the second pore occupancy image, the third pore occupancy image and the fourth pore occupancy image;
the data processing comprises determining a volumetric fraction of first fluid or/and a volumetric fraction of second fluid as a function of pore size from the pore size distribution as a function of pore size, from the pore distribution of pores occupied by the first fluid as a function of pore size, and/or from pore distribution of pores occupied by the second fluid as a function of pore size;
the data processing comprises determining a spatial correlation of wettability using at least one of the first pore occupancy image, the second pore occupancy image, the third pore occupancy image and the fourth pore occupancy image;
determining a spatial correlation of wettability comprises establishing a variogram function relating a variance parameter to a distance and determining from the variogram function at which distance the variance parameter becomes stationary, the spatial correlation being said distance;
the wettability parameter is a wettability scheme such as first fluid-wet, second fluid-wet or mixed-wet, a pore network wettability feature such as a radius/center of first fluid-wet and second fluid-wet pores, a wettability model such as Mixed-Wet Small,
Mixed-Wet Large or Fractional-Wet model, a wettability correlation parameter, such as a spatial wettability correlation length, and/or a fraction of first fluid-wet and/or second fluid-wet pores.

The invention also concerns a pore scale simulation process, carried out with a computer, the process comprising the following steps:
acquiring and segmenting a porous sample into images of sections of the sample;
carrying out pore scale flow modelling simulations, in particular dynamic porous network flow modelling simulations, using a model obtained from the acquiring and segmenting of the porous sample and at least a wettability parameter determined by the determining method as defined above;
determining at least a macroscopic parameter, in particular a petrophysical parameter, using the pore scale flow modelling simulation.

The simulation process according to the invention may comprise the following feature:
it comprises, after acquiring and segmenting the porous sample, extracting a reconstructed calculated porous network into an assembly of pore bodies connected through pore throats from the acquiring and segmenting of the porous sample, the pore scale modelling simulations being carried out in the in the reconstructed porous network.

### BRIEF DESCRIPTION OF THE DRAWINGS

The invention also relates to a porous sample wettability parameter determining system configured to carry out a determining method as disclosed above.

The invention will be better understood, based on the following description, taken solely as an example, are made in reference to the following drawings, in which:

FIG. 1 is a schematic view of a system configured for carrying out a porous sample wettability parameter determining method according to the invention;

FIGS. 2a, 2b, 3a and 3b are images and histograms respectively obtained FIG. 2a after saturating the porous sample with a first phase fluid, FIG. 2b after carrying out a spontaneous imbibition of the first open face with a second phase fluid for a first given time, FIG. 3a after saturating the porous sample with the second phase fluid, FIG. 3b after carrying out a spontaneous drainage of the second open face with the first phase fluid for a second given time;

FIG. 4 is a graph depicting a saturation in the second phase fluid in successive sections taken along a first end section of the porous sample respectively after saturating the porous sample with a first phase fluid, after carrying out a spontaneous imbibition of the first open face with a second phase fluid for a first given time, and a corresponding saturation difference;

FIG. 5 is a graph depicting a saturation in the first phase fluid in successive sections taken along a second end section of the porous sample respectively after saturating the porous sample with a second phase fluid, and after carrying out a spontaneous drainage of the second open face with the first phase fluid for a second given time;

FIG. 6 is a graph illustrating a second phase fluid wet volumetric ratio as a function of pore radius;

FIG. 7 is a graph illustrating pore radii distribution and second phase fluid wet pore radii distribution;

FIG. 8 is a graph illustrating a variogram plot for the second phase fluid wet pores;

FIG. 9 is a scheme illustrating the successive steps of a digital porous sample physics simulation process carried out using wettability parameters obtained by the method according to the invention.

### DETAILED DESCRIPTION

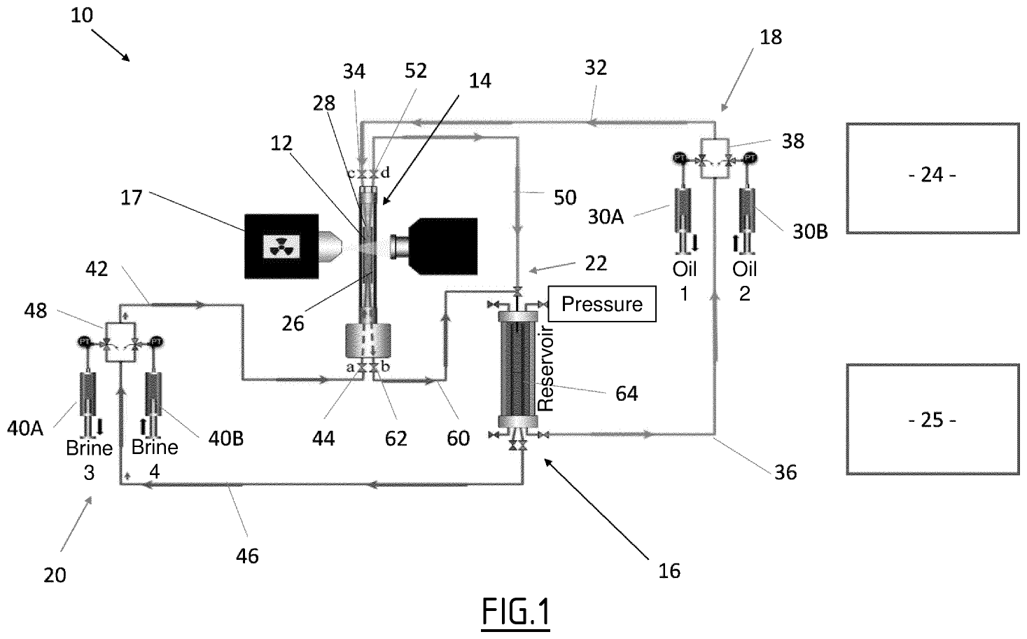

An example of a determining method according to the invention is advantageously carried out in the determining system 10 shown in FIG. 1. The method is intended for determining at least a wettability parameter of a porous sample 12, associated with a first fluid and a second fluid.

The porous sample 12 is for example a formation sample extracted from a sub-soil. The formation sample is in particular a rock sample having an internal porosity or another porous sample. It defines pores having an internal volume.

The pores of the sample may be occupied by a first fluid having a first phase, in particular an oil phase fluid, for example oil, by a second fluid having a second phase, in particular a water phase fluid such as brine, or by a mixture of first fluid and second fluid, some of the pores being wet with the first fluid, other pores being wet with the second fluid.

More generally, the first fluid or the second fluid can be a gas such as CO2 or any other fluid phase.

The ratio of the volume of pores occupied by a particular fluid to the total volume of pores in a region of the porous sample or in the whole porous sample is referred to as the saturation S of said particular fluid in the region of the porous sample or in the whole porous sample.

Typically, the porous sample 12 is cylindrical, with a circular cross-section.

The diameter of the porous sample 12 is generally comprised between 1 mm and 10 mm. Its length is for example comprised between 10 mm and 500 mm.

Wettability parameters determined by the method according to the invention are for example a the wettability scheme of the porous sample (e.g. first fluid-wet, in particular oil-wet, second fluid-wet, in particular water wet, mixed-wet), the spatial correlation length of the wettability, the potential wettability correlation to the radii and, as a consequence, the type of wettability model (e.g. Mixed-Wet Small, Mixed-Wet Large or Fractional-Wet), and the first fluid or second fluid saturation at zero capillary pressure, in particular the water saturation at zero capillary pressure that is useful to determine the first fluid-wet and second fluid-wet fractions.

As shown in FIG. 1, the determining system 10 for carrying out the method according to the invention comprises a pressurized flow cell 14 receiving the porous sample 12, a fluid reservoir 16 coupled to the flow cell 14, and an scanner 17 configured to take fluid phase sensitive scans of sections of the porous sample 12 in the flow cell 14 to determine pore occupancy images of the sections of the porous sample.

The determining system 10 further comprises a first fluid distributor 18, a second fluid distributor 20, and a fluid collector 22.

The determining system 10 comprises a controller 24 configured to control the scanner 17, the first fluid distributor 18, the second fluid distributor 20, and the fluid collector 22 to carry out the steps of the method according to the invention, and a data analyzer 25 configured to process data extracted from the pore occupancy images obtained with the scanner 17 to determine at least a wettability parameter of the porous sample.

The flow cell 14 comprises an outer wall able to confine the lateral face of the porous sample 12 and to hold the porous sample 12 under pressure, for example at a pressure comprised between 1 bar and 100 bar.

In the flow cell 14, the porous sample 12 defines a first open face 26, here its lower transverse end face and a second opposed open face 28, here its upper transverse end face. The first open face 26 is connected to the second fluid distributor 20 to receive second fluid. The second open face 28 is connected to the first fluid distributor 18 to receive first fluid.

In this example, the first fluid distributor 18 comprises a first syringe pump 30A and preferentially a second syringe pump 30B configured to alternatively receive first fluid from the reservoir 16 and distribute first fluid to the flow cell 14. It comprises a first fluid distribution pipe 32 equipped with a control valve 34 connected to the second face 28 of the porous sample 12 in the flow cell 14, a first fluid feeding pipe 36 connected to the reservoir 16 and a selective connector 38 selectively connecting the first syringe pump 30A and the second syringe pump 30B respectively to the first fluid distribution pipe 32 and to the first fluid feeding pipe 36.

Thanks to the alternate operation of the first syringe pump 30A and of the second syringe pump 30B, via the selective connector 38, first fluid can be continuously fed from the reservoir 16 to either one of the syringe pumps 30A, 30B via the first fluid feeding pipe 36, while the other one of the syringe pumps 30A, 30B distributes first fluid to the flow cell 14 via the first fluid distribution pipe 32.

In this example, the second fluid distributor 20 comprises a first syringe pump 40A and preferentially a second syringe pump 40B configured to alternatively receive second fluid from the reservoir 16 and distribute second fluid to the flow cell 14. It comprises a second fluid distribution pipe 42 equipped with a control valve 44 connected to the first face 26 of the porous sample 12 in the flow cell 14, a second fluid feeding pipe 46 connected to the reservoir 16 and a selective connector 48 selectively connecting the first syringe pump 40A and the second syringe pump 40B respectively to the second fluid distribution pipe 42 and to the second fluid feeding pipe 46.

Thanks to the alternate operation of the first syringe pump 40A and of the second syringe pump 40B, via the selective connector 48, second fluid can be continuously fed from the reservoir 16 to either one of the syringe pumps 40A, 40B via the second fluid feeding pipe 46, while the other one of the syringe pumps 40A, 40B distributes second fluid to the flow cell 14 via the second fluid distribution pipe 32.

The fluid collector 22 comprises a first collection pipe 50, equipped with a control valve 52, the first collection pipe 50 connecting the second face 28 of the porous sample 12 in the flow cell 14 to the reservoir 16. The fluid collector 22 also comprises a second collection pipe 60 equipped with a control valve 62 connecting the first face 26 of the porous sample 12 in the flow cell 14 to the reservoir 16.

The reservoir 16 comprises an inner separator 64 configured to collect fluid from the first collection pipe 50 and from the second collection pipe 60 and to separate it into first fluid and second fluid to respectively feed the first fluid feeding pipe 36 with first fluid and the second fluid feeding pipe 46 with second fluid.

The controller 24 is configured to control the control valves 34, 52 and 44, 62 and the respective syringe pumps 30A, 30B and 40A, 40B to selectively carry out forced drainage, forced imbibition, spontaneous drainage, or spontaneous imbibition as it will be described below. The controller 24 for example comprises a computer having a processor and a memory comprising software modules configured to carry out the control of the scanner 17, of the first fluid distributor 18, of the second fluid distributor 20, and of the fluid collector 22.

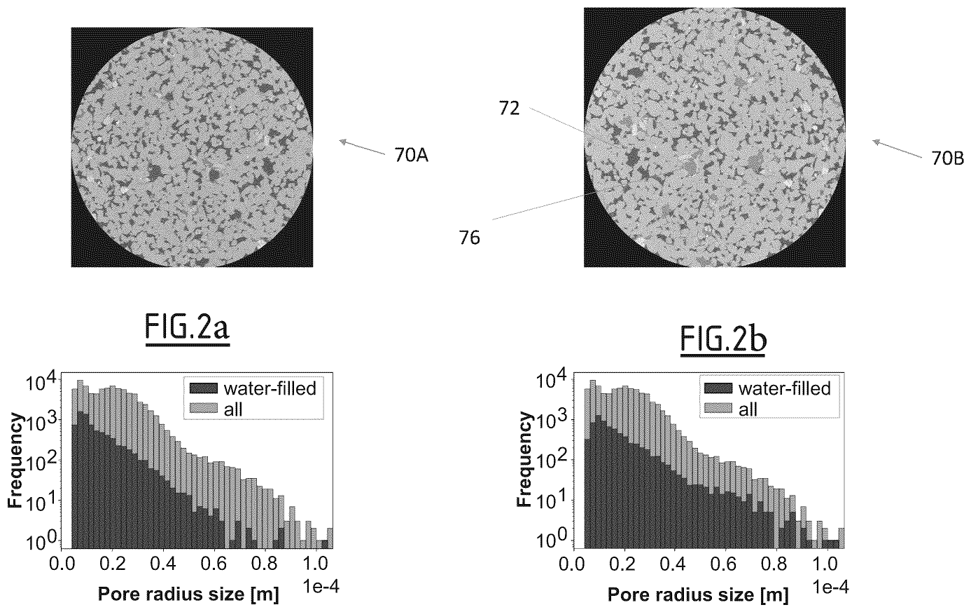

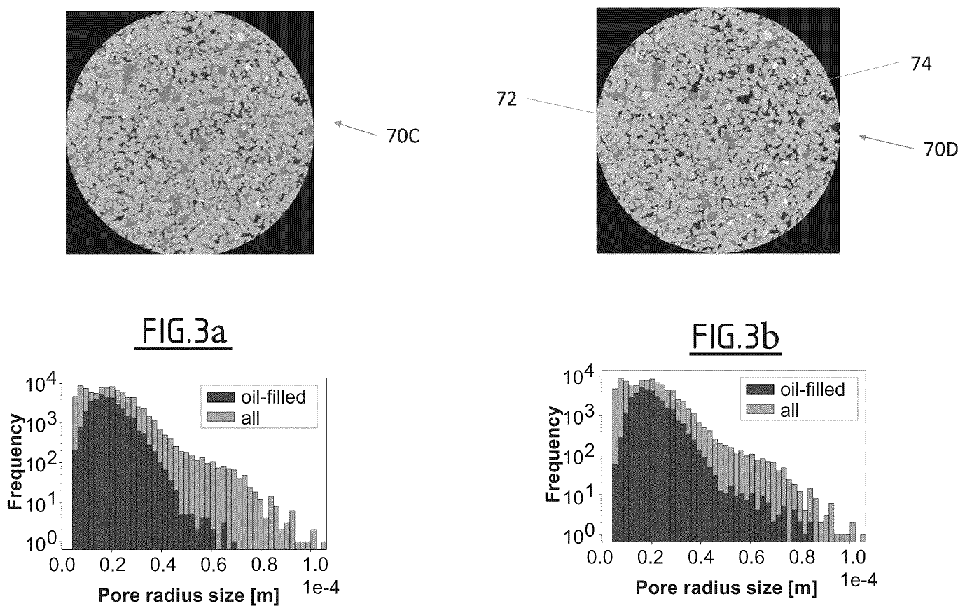

The scanner 17 is for example a micro computerized tomography (CT) scanner or an X-ray scanner. It is configured to take fluid sensitive high resolution scans of successive transverse sections of the porous sample 12 within the flow cell 14 to obtain pore occupancy images 70A to 70D of each section as shown in FIGS. 2 and 3. “High resolution” means a resolution advantageously in the micron range (i.e. less than 1 mm) or in the submicron range (i.e. less than 1 μm).

As visible in FIGS. 2 and 3, the pore occupancy images 70A to 70D show the rock network structure 72, the pores 74 wet with first fluid and the pores 76 wet with second fluid with different levels of gray, allowing a discrimination among these volumes.

The analyzer 25 is configured to process the pore occupancy images 70A to 70D obtained from the scans taken by the scanner 17 to determine the wettability properties, as it will be described below. The analyzer for example comprises a computer having a processor and a memory comprising software modules configured to carry out the data processing from the pore occupancy images 70A to 70D.

An example of determining method according to the invention will now be described.

Initially, in reference to FIG. 1, the determining method comprising placing the porous sample 12 in the flow cell 14. As described above, the porous sample 12 has a first open face 26 connected to the second fluid distributor 20 and a second open face 28 connected to the first fluid distributor 18.

The determining method then comprises saturating the porous sample 12 with the first fluid, in particular an oil phase fluid, until the first fluid occupies a majority of the pore space (i.e. more than 50% in volume of the total volume of the pores).

Accordingly, the controller 24 opens control valves 34 and 62, and closes control valves 44 and 52. It activates syringe pumps 30A, 30B to distribute first fluid from the reservoir 16 to the second face 28 of the porous sample 12 and generate a first forced drainage of the porous sample 12 with first fluid. The recovered fluid is collected via the second collection pipe 60 and is send to the reservoir 16 to be separated and recycled.

When the porous sample 12 is saturated with the first fluid by viscous displacement or porous plate permeation, only residual second fluid remains in the porous sample at an initial second fluid saturation (Swi in the case of a water phase fluid).

In a particular embodiment, mineral oil (such as Marcol52) is used followed by replacing the mineral oil with toluene, then with dead crude oil. Advantageously, the porous sample 12 is aged for at least a week at a temperature of at least 50° C., in particular between 70° C. and 90° C. Advantageously, this is followed by injection of more than 2 Pore volumes of decaline to remove the dead oil and by injection of mineral oil (such as Marcol52) to replace decaline. Fluid replacement is carried out at low flow rates to ensure the initial water saturation is not changed.

The determining method then comprises taking a fluid sensitive first high resolution scan to determine a first pore occupancy image 70A of at least one first transverse section of the porous sample 12. Advantageously, the controller 24 pilots the scanner 17 to take several first scans to obtain first pore occupancy images 70A of several successive first sections of the porous sample 12 along at least a first end region of the porous sample 12. The first pore occupancy images 70A are transmitted to the analyzer 25 to be stored and processed.

The determining method then comprises, after taking each first scan, carrying out a spontaneous imbibition of the first open face 26 with a second fluid, in particular a water phase fluid, for a first given time T1.

Accordingly, the controller 24 opens control valves 44 and 62, and closes control valves 34 and 52. It activates syringe pumps 40A, 40B to distribute second fluid from the reservoir 16 to the first face 26 of the porous sample 12 and generate the spontaneous imbibition of the porous sample 12 with second fluid. The second fluid is leached at counter current along the first open face 26 without forcing the second fluid in the porous sample 12.

In particular, a very low capillary number (for example smaller than 10⁻⁸, in particular 8×10⁻⁹) may be used for the leaching to remove the first fluid from the porous sample 12 without forcing the flow of second fluid into the porous sample 12.

The recovered fluid is collected after leaching via the second collection pipe 60 and is send to the reservoir 16 to be separated and recycled.

The first given time T1 during which spontaneous imbibition of the second fluid occurs is at least one day, in particular more than one week and preferentially between one week and ten weeks.

The determining method then comprises taking a fluid sensitive second high resolution scan to determine a second pore occupancy image 70B of the or each first section of the porous sample, corresponding to the or each first pore occupancy image 70A taken previously. Advantageously, the controller 24 pilots the scanner 17 to take several second scans to obtain second pore occupancy images 70B of the several successive first sections of the porous sample 12 along at least the first end region of the porous sample 12. The second pore occupancy images 70B are transmitted to the analyzer 25 to be stored and processed.

The determining method then comprises, after taking each second scan, saturating the porous sample 12 with the second fluid, until the second fluid occupies a majority of the pore space (i.e. more than 50% in volume of the total volume of the pores).

Accordingly, the controller 24 opens control valves 44 and 52, and closes control valves 34 and 62. It activates syringe pumps 40A, 40B to distribute second fluid from the reservoir 16 to the first face 26 of the porous sample 12 and generate a first forced imbibition of the porous sample 12 with second fluid. The recovered fluid is collected via the first collection pipe 50 and is send to the reservoir 16 to be separated and recycled.

When the porous sample 12 is saturated with the second fluid by viscous displacement, only residual first fluid remains at a residual first fluid saturation (So in the case of an oil phase fluid).

The determining method then comprises taking a fluid sensitive third high resolution scan to determine a third pore occupancy image 70C of at least one second section of the porous sample. Advantageously, the controller 24 pilots the scanner 17 to take several third scans to obtain third pore occupancy images 70C of several successive second sections of the porous sample 12 along at least a second end region of the porous sample 12. The third pore occupancy images 70C are transmitted to the analyzer 25 to be stored and processed.

The determining method then comprises carrying out a spontaneous drainage of the second open face 28 of the porous sample 28 with the first fluid for a second given time T2.

Accordingly, the controller 24 opens control valves 34 and 52, and closes control valves 44 and 62. It activates syringe pumps 30A, 30B to distribute first fluid from the reservoir 16 to the second face 28 of the porous sample 12 and generate the spontaneous drainage of the porous sample 12 with first fluid. The first fluid is leached at counter current along the second open face 28 without forcing the first fluid in the porous sample 12.

In particular, a very low capillary number ((for example smaller than 10⁻⁸, in particular 8×10⁻⁹)) may be used for the leaching to remove the second fluid from the porous sample 12 without forcing the flow of first fluid into the porous sample 12.

The recovered fluid is collected after leaching via the first collection pipe 50 and is send to the reservoir 16 to be separated and recycled.

The second given time T2 during which spontaneous drainage occurs is substantially equal to the first given time T1 during which spontaneous imbibition occurs. For example, the absolute value of the difference between the first given time T1 and the second given time T2 is smaller than 10%.

The determining method then comprises taking a taking a fluid sensitive fourth high resolution scan to determine a fourth pore occupancy image 70D of the or each second section of the porous sample 12 corresponding to the or each third pore occupancy image 70D obtained previously. Advantageously, the controller 24 pilots the scanner 17 to take several fourth scans to obtain fourth pore occupancy images 70D of the several successive second sections of the porous sample 12 along at least the second end region of the porous sample 12. The fourth pore occupancy images 70B are transmitted to the analyzer 25 to be stored and processed.

The method then comprises processing data extracted from each first pore occupancy image 70A, each second pore occupancy image 70B, each third pore occupancy image 70C and each fourth pore occupancy image 70D with the analyzer 25 to determine at least a wettability parameter of the porous sample 12.

The analyzer 25 processes the pore occupancy images 70A to 70D to determine the surface areas of the rock network structure 72, of the pores 74 wet with first fluid and of the pores 76 wet with second fluid. This allows determining a local unitary volume (by multiplication by a unitary length) of each pore 74, 76, of each pore 74 wet with first fluid and of each pore 76 wet with second fluid, at each position along the porous sample 12 at which a pore occupancy image 70A to 70D has been taken, in particular in the first and second end regions.

The data processing advantageously includes the analyzer 25 calculating a saturation in first fluid or/and in second fluid in each first pore occupancy image 70A, each second pore occupancy image 70B, each third pore occupancy image 70C and each fourth pore occupancy image 70D.

Based on the data processing, the analyzer 25 determines the wettability scheme (e.g second fluid-wet, first fluid-wet or mixed-wet)

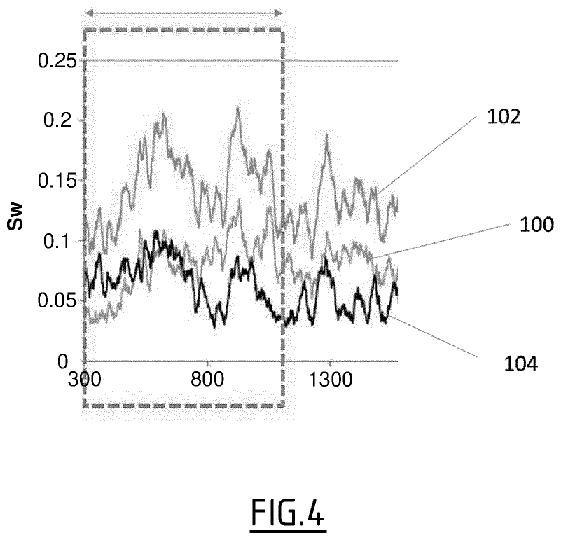

As shown in FIG. 4, the saturations in the second fluid in the first end section of the porous sample (corresponding for example to more than 100 images 70A, 70B, for example between 700 and 900 images) are plotted as a function of the longitudinal position along the first end section of the porous sample. Curve 100 corresponds to the initial saturation in second fluid (Swi), curve 102 corresponds to the saturation in second fluid after spontaneous imbibition, and curve 104 corresponds to the difference in saturation at each position before and after spontaneous imbibition.

Since the water saturation increased (here by 7.5% along the inlet side and by 1.5% on the same length from the not shown outlet side), the analyzer 25 determines that the porous sample is a mixed-wet sample.

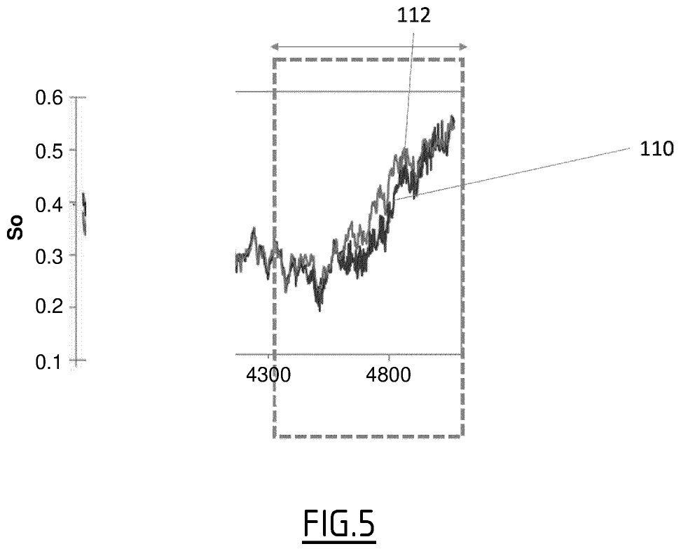

As shown in FIG. 5, the saturations in the first fluid in the second end section of the porous sample (corresponding for example to more than 100 images 70C, 70D, for example between 700 and 900 images) are plotted as a function of the longitudinal position along the second end section of the porous sample 12. Curve 110 corresponds to the residual saturation in first fluid (So), and curve 112 corresponds to the saturation in first fluid after spontaneous drainage.

From the saturation profile of the first fluid in FIG. 5 which shows a capillary end effect, the analyzer 25 determines an indication of first fluid-wet part in the porous sample 12.

Furthermore, the analyzer 25 may calculate the oil saturation at Pc equal to zero at the end of the porous sample (here equal to 55%). This helps characterizing the first fluid-wet fraction in the system that is needed to achieve such a saturation. In the present case, it is estimated 53-63% of first fluid-wet fraction after a fast PNM sensitivities study where the Pc=0 was reached at Sw˜0.45.

Finally, the fact that the imbibed water volume was much higher than the imbibed oil volume for the same duration and at the same sample length gives an indication that in a considerable amount of the first fluid-wet pores the receding contact angle was lower than 90°.

Advantageously, the data processing includes calculating a pore size distribution as a function of pore size, a pore distribution of pores occupied by the first fluid as a function of pore size, and/or a pore distribution of pores occupied by the second fluid as a function of pore size, in each first pore occupancy image 70A, in each second pore occupancy image 70B, in each third pore occupancy image 70C and in each fourth pore occupancy image 70D.

In a particular embodiment, the analyzer 25 carries out a pore network extraction to determine the radii/centers of each first fluid-wet and second fluid-wet pore.

FIGS. 2a, 2b, 3a and 3b show examples of histograms classifying the frequency of occurrence of a pore and/or pores occupied by the first fluid (FIGS. 3a and 3b), by the second fluid (FIGS. 2a and 2b) as a function of pore size, obtained by the analyzer 25.

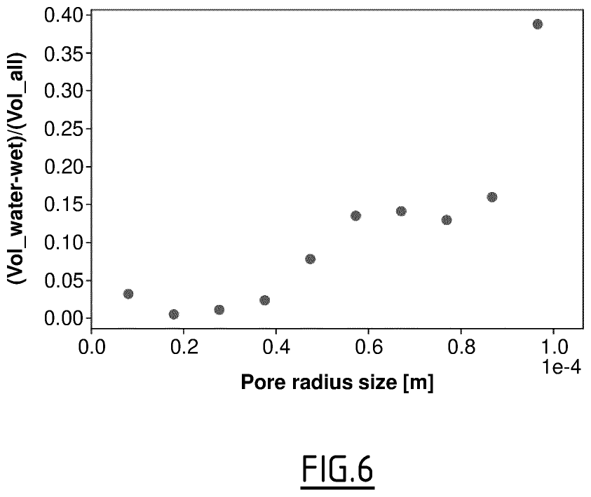

As shown in FIG. 6, the volumetric fraction of second fluid-wet pores as a function of the pore radii is calculated by the analyzer to identify a wettability model among a Mixed-Wet Small, Mixed-Wet Large or Fractional-Wet model. In the particular example of FIG. 6, a clear correlation is observed between the pore radius and the percentage of second fluid-wet pores in the system that demonstrates that for this system large pores are more likely to be second fluid-wet. A Mixed-Wet Small wettability model is thus determined.

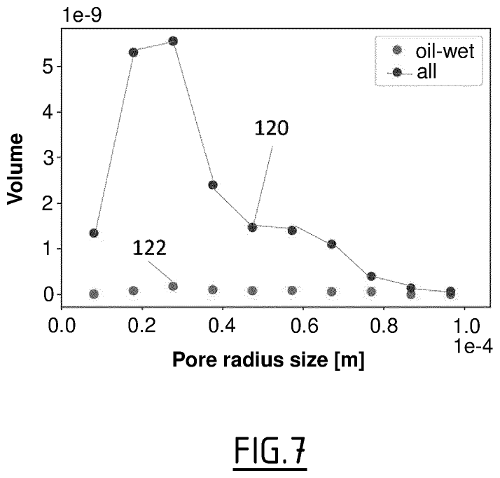

The data processing may comprise determining a volumetric fraction of first fluid or/and a volumetric fraction of second fluid as a function of pore size from the pore size distribution as a function of pore size, from the pore distribution of pores occupied by the first fluid as a function of pore size, and/or from pore distribution of pores occupied by the second fluid as a function of pore size. As shown in FIG. 7, the analyzer 25 here determines the global pore radii distribution (curve 120) and the second fluid-wet pore size distribution (curve 122).

In an embodiment, the data processing comprises determining a spatial correlation of wettability using each first pore occupancy image 70A, each second pore occupancy image 70B, each third pore occupancy image 70C or/and each fourth pore occupancy image 70D.

The analyzer 25 establishes a variogram function relating a variance parameter, here a semivariance to a distance and determining from the variogram function at which distance the variance parameter becomes stationary, the spatial correlation being said distance.

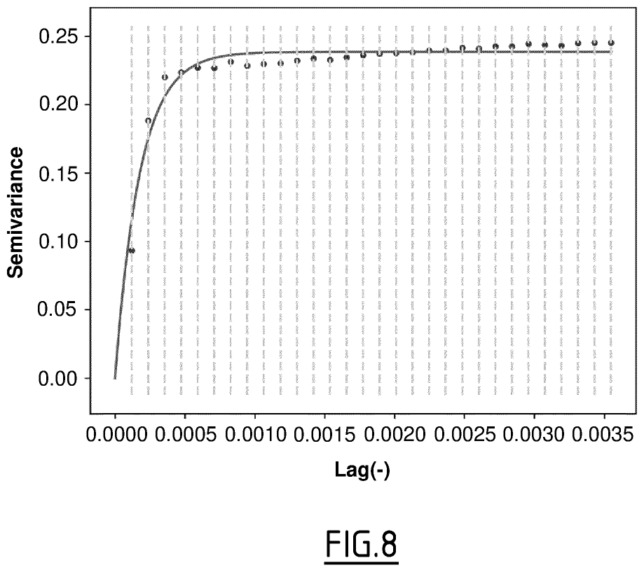

In the example of FIG. 8, the varigram plot is calculated for the second fluid phase pores advantageously using the method disclosed in the article by Malicke M. SciKit-GStat 1.0: a SciPy-flavored geostatistical variogram estimation toolbox written in Python. Geoscientific Model Development 2022; 15(6): 2505-32. The correlation length is estimated from the onset of the constant slope of the curve, here at 500 μm.

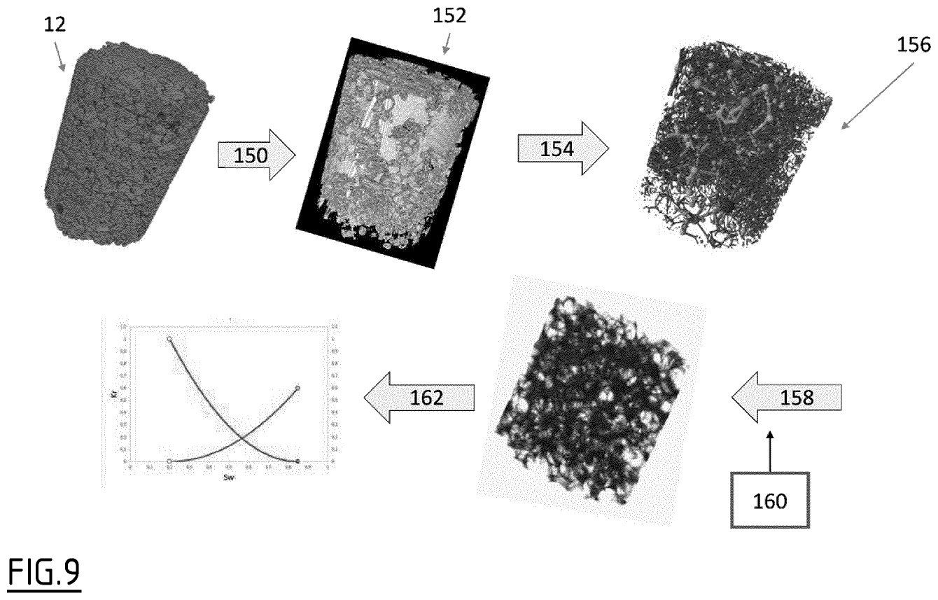

A digital porous sample physics simulation process, carried out with at least one computer, will be now described, as illustrated in FIG. 9.

The process is carried out in a computer having a processor and a memory comprising software modules configured to carry out the steps of the method.

The process first comprises a step 150 of acquiring and segmenting a porous sample 12 into images 152 of sections of the porous sample. This is done by Acquiring micro-CT images of the porous sample 12 potentially along with an image resolution enhancing program using a treatment method such as described in Wang X, Yu K, Wu S, Gu J, Liu Y, Dong C, Qiao Y, Change Loy C. Esrgan: Enhanced super-resolution generative adversarial networks. In: Proceedings of the European conference on computer vision (ECCV) workshops.

It then comprises a step 154 of extracting from the acquisition and segmentation images, a reconstructed calculated porous network 156 into an assembly of pore bodies connected through pore throats.

Various algorithms exist to extract the skeleton of the 3D model that carries the essential geometric and topological information of the underlying pore system. Advantageously a pore network extraction platform called GNextract developed in Raeini A Q, Bijeljic B, Blunt M J. Generalized network modeling: Network extraction as a coarse-scale discretization of the void space of porous media. Phys. Rev. E 2017; 96(1): 1331, may be used.

The process then comprises a step 158 of carrying out quasi-static porous network flow modelling simulations in the reconstructed porous network. The simulation is for example carried out via a model disclosed in Regaieg M, Moncorgé A., Adaptive dynamic/quasi-static pore network model for efficient multiphase flow simulation. Computational Geosciences 2017; 21(4): 795-806.

According to the invention, the model is initialized with at least a wettability parameter 160 determined by the determining method disclosed above.

The process then comprises a step 162 of determining at least a petrophysical parameter using the porous network flow modelling simulations of step 158. Advanced rock properties such as relative permeability and capillary pressure can for example be computed.

Thanks to the use of pore occupancy images 70A, 70B, 70C, 70D taken at the end of each of the spontaneous phases to determine the new invasions, the determining method according to the invention allows a determination of numerous wettability parameters needed by dynamic porous network flow modelling simulations (in particular wettability scheme, correlation to radii, spatial correlation and fractions of oil wet-water wet pores) from a single series of steps.

The determining method according to the invention is fast. It has less bias due to image resolution and is consequently more precise. It provides richer wettability information to the simulation comparing to known methods.

In a variant, Direct Numerical Simulation, or Lattice Boltzmann method are used in replacement of Pore Network Modelling.

In another variant, the imbibitions and drainages are carried out from the same open face of the porous sample 12.

1. A porous sample wettability parameter determining method, comprising:
placing a porous sample in a flow cell, the porous sample having a first open face and a second open face and an internal pore space;
saturating the porous sample with at least one first fluid until the at least one first fluid occupies a majority of the pore space;
taking a fluid sensitive first high resolution scan of at least one first section of the porous sample to determine a first pore occupancy image of the at least one first section of the porous sample;
after taking the first scan, carrying out a spontaneous imbibition from the first open face with a least one second fluid for a first given time;
taking a fluid sensitive second high resolution scan of the at least one first section of the porous sample to determine a second pore occupancy image of the at least one first section of the porous sample;
after taking the second scan, saturating the porous sample with the at least one second fluid until the at least one second fluid occupies at least a majority of the pore space;
taking a fluid sensitive third high resolution scan of at least one second section of the porous sample to determine a third pore occupancy image of the at least one second section of the porous sample;
after taking the third scan, carrying out a spontaneous drainage with the first fluid from the second open face for a second given time,
taking a fluid sensitive fourth high resolution scan of the at least one second section of the porous sample to determine a fourth pore occupancy image of the at least one second section of the porous sample;
processing data extracted from the first pore occupancy image, the second pore occupancy image, the third pore occupancy image and the fourth pore occupancy image to determine at least a wettability parameter associated with the porous sample.

2. The determining method according to claim 1, wherein carrying out the spontaneous imbibition of the first open face with a second fluid comprises leaching the second fluid along the first open face without forcing the second fluid in the porous sample, carrying out a spontaneous drainage of the first open face with the first fluid comprising leaching the first fluid along the second open face without forcing the first fluid in the porous sample.

3. The determining method according to claim 1, wherein the first open face and the second open face are opposite faces of the porous sample or are a same face of the porous sample.

4. The determining method according to claim 1, wherein taking a fluid sensitive first, second, third and fourth high resolution scan comprises taking a computed tomography image or a X-ray image of a section of the porous sample.

5. The determining method according to claim 1, wherein taking a fluid sensitive first high resolution scan of at least one first section of the porous sample comprises taking several fluid sensitive first high resolution scans of several successive first sections of the porous sample along at least a first end region of the porous sample, taking a fluid sensitive second high resolution scan of the at least one first section of the porous sample comprises taking several fluid sensitive second high resolution scans of several successive first sections of the porous sample along at least the first end region of the porous sample, and wherein taking a fluid sensitive third high resolution scan of at least one second section of the porous sample comprises taking several fluid sensitive third high resolution scans of several successive second sections of the porous sample along at least a second end region of the porous sample, taking a fluid sensitive fourth high resolution scan of the at least one second section of the porous sample comprises taking several fluid sensitive fourth high resolution scans of several successive second sections of the porous sample along at least the second end region of the porous sample.

6. The determining method according to claim 1, wherein the at least one first section is located in the vicinity of the first open face, the at least one second section being located in the vicinity of the second open face.

7. The determining method according to claim 1, wherein the difference between the first given time and the second given time is smaller than 10%.

8. The determining method according to claim 1, wherein the data processing includes calculating a saturation in first fluid or/and in second fluid in the first pore occupancy image, the second pore occupancy image, the third pore occupancy image and the fourth pore occupancy image.

9. The determining method according to claim 8, wherein the data processing comprises calculating a difference between a saturation in first fluid or/and in second fluid in the second pore occupancy image and the corresponding saturation in first fluid or/and in second fluid in the first pore occupancy image, and/or the data processing comprises calculating a difference between a saturation in first fluid or/and in second fluid in the fourth pore occupancy image and the corresponding saturation in first fluid or/and in second fluid in the third pore occupancy image.

10. The determining method according to claim 1, wherein the data processing includes calculating a pore size distribution as a function of pore size and/or a pore distribution of pores occupied by the first fluid as a function of pore size, and/or pore distribution of pores occupied by the second fluid as a function of pore size, in the first pore occupancy image, the second pore occupancy image, the third pore occupancy image and the fourth pore occupancy image.

11. The determining method according to claim 10, wherein the data processing comprises determining a volumetric fraction of first fluid or/and a volumetric fraction of second fluid as a function of pore size from the pore size distribution as a function of pore size, from the pore distribution of pores occupied by the first fluid as a function of pore size, and/or from pore distribution of pores occupied by the second fluid as a function of pore size.

12. The determining method according to claim 1, wherein the data processing comprises determining a spatial correlation of wettability using at least one of the first pore occupancy image, the second pore occupancy image, the third pore occupancy image and the fourth pore occupancy image.

13. The determining method according to claim 12, wherein determining a spatial correlation of wettability comprises establishing a variogram function relating a variance parameter to a distance and determining from the variogram function at which distance the variance parameter becomes stationary, the spatial correlation being said distance.

14. A pore scale simulation process, carried out with a computer, the process comprising:
acquiring and segmenting a porous sample into images of sections of the sample;
carrying out pore scale flow modelling simulations using a model obtained from the acquiring and segmenting of the porous sample and at least a wettability parameter determined by the method according to claim 1;
determining at least a macroscopic parameter using the pore scale flow modelling simulation.

15. The pore scale simulation process of claim 14, comprising, after acquiring and segmenting the porous sample, extracting a reconstructed calculated porous network into an assembly of pore bodies connected through pore throats from the acquiring and segmenting of the porous sample, the pore scale modelling simulations being carried out in the in the reconstructed porous network.

16. A porous sample wettability parameter determining system configured to carry out a method according to claim 1.
